(19) United States
(12) Patent Application Publication
(10) Pub. No.: US 2020/0110923 A1
(43) Pub. Date: Apr. 9, 2020

(54) QUANTITATIVE EVALUATION APPARATUS

(71) Applicant: OLYMPUS CORPORATION, Tokyo (JP)

(72) Inventors: Yohei TANIKAWA, Tokyo (JP); Shinichi TAKIMOTO, Tokyo (JP); Shintaro TAKAHASHI, Tokyo (JP); Masaru MIZUNAKA, Tokyo (JP); Akira MATSUSHITA, Tokyo (JP); Tsuyoshi MOCHIZUKI, Tokyo (JP); Koh MOHRI, Tokyo (JP)

(73) Assignee: OLYMPUS CORPORATION, Tokyo (JP)

(21) Appl. No.: 16/707,498

(22) Filed: Dec. 9, 2019

Related U.S. Application Data
(63) Continuation of application No. PCT/JP2017/022063, filed on Jun. 15, 2017.

(51) Int. Cl. G06K 9/00; C12M 1/34

(52) U.S. Cl. CPC G06K 9/00134; C12M 41/36

(57) ABSTRACT

A quantitative evaluation apparatus of the present invention includes: a counter that counts the numbers of cells or densities of cells contained in individual images of a plurality of image-capturing regions in a culturing vessel for culturing the cells; an evaluation-value calculating portion that calculates an evaluation value related to variation in the distribution of the cells in the culturing vessel on the basis of the numbers of the cells or the densities of cells counted by means of the counter in each of the images; a determining portion that determines whether or not the evaluation value calculated by the evaluation-value calculating portion is in a range of a prescribed threshold; and a display that displays determination result determined by the determining portion.

### CROSS-REFERENCE TO RELATED APPLICATIONS

This is a continuation of International Application PCT/JP2017/022063, with an international filing date of Jun. 15, 2017, which is hereby incorporated by reference herein in its entirety.

### TECHNICAL FIELD

The present invention relates to a quantitative evaluation apparatus.

### BACKGROUND ART

In cell culturing, it is necessary to regularly perform medium replacement, in which an old medium is replaced with a new medium, and passaging, in which proliferated cells are transferred to another culturing vessel after reducing the number of cells by means of dilution. In passaging, it is preferable that cells be seeded in the culturing vessel in a uniformly dispersed manner. In the related art, there is a known cell-culturing apparatus in which cells in a culturing vessel are uniformly dispersed as much as possible (for example, see Japanese Unexamined Patent Application, Publication No. 2010-268813).

### SUMMARY OF INVENTION

An aspect of the present invention is a quantitative evaluation apparatus including: a counter that counts numbers of cells or densities of cells contained in individual images of a plurality of image-capturing regions in a culturing vessel for culturing the cells; an evaluation-value calculating portion that calculates an evaluation value related to variation in distribution of the cells in the culturing vessel on the basis of the numbers of the cells or the densities of the cells counted by means of the counter in each of the images; a determining portion that determines whether or not the evaluation value calculated by the evaluation-value calculating portion is in a range of a prescribed threshold; and a display that displays determination result determined by the determining portion.

### BRIEF DESCRIPTION OF DRAWINGS

FIG. 1 is a configuration diagram showing, in outline, a quantitative evaluation apparatus according to a first embodiment of the present invention.

FIG. 2 is a diagram showing an example of a culturing vessel in which the distribution state of cells is confirmed by the quantitative evaluation apparatus in FIG. 1.

FIG. 3 is a diagram showing another example of a culturing vessel in which the distribution state of cells is confirmed by the quantitative evaluation apparatus in FIG. 1.

FIG. 4A is a diagram showing an example of a screen of a notifying portion for setting a threshold in the quantitative evaluation apparatus in FIG. 1.

FIG. 4B is a diagram showing an example of an image-capturing start button for setting image capturing to a multiple-image-capturing mode in the quantitative evaluation apparatus in FIG. 1.

FIG. 4C is a diagram showing an example of a screen of the notifying portion for issuing an instruction for acquiring an image in the quantitative evaluation apparatus in FIG. 1.

FIG. 4D is a diagram showing an example of a screen of the notifying portion for displaying an evaluation result of the distribution state of cells in the quantitative evaluation apparatus in FIG. 1.

FIG. 5 is a flowchart for explaining steps for evaluating the distribution state of cells by means of the quantitative evaluation apparatus in FIG. 1.

FIG. 6 is a diagram showing an example of the relationship between the range of a prescribed threshold and the ratios of the numbers of cells in individual image-capturing regions.

FIG. 7 is a configuration diagram showing, in outline, a quantitative evaluation apparatus according to a second embodiment of the present invention.

FIG. 8A is a diagram showing an example of an image-capturing start button for setting image capturing to a multiple-image-capturing mode in the quantitative evaluation apparatus in FIG. 7.

FIG. 8B is a diagram showing an example of a screen of a notifying portion for issuing an instruction for acquiring an image in the quantitative evaluation apparatus in FIG. 7.

FIG. 8C is a diagram showing an example of a screen of the notifying portion for prompting a user to confirm the focus position in the quantitative evaluation apparatus in FIG. 7.

FIG. 8D is a diagram showing an example of a screen of the notifying portion for displaying an evaluation result of the distribution state of cells in the quantitative evaluation apparatus in FIG. 7.

FIG. 9 is a flowchart for explaining steps for evaluating the distribution state of cells by means of the quantitative evaluation apparatus in FIG. 7.

FIG. 10 is a diagram showing an example of a cell suspension state immediately after the cells are seeded in a culturing vessel.

FIG. 11 is a configuration diagram showing, in outline, a quantitative evaluation apparatus according to a third embodiment of the present invention.

FIG. 12A is a diagram showing an example of an image-capturing start button for setting image capturing to a multiple-image-capturing mode in the quantitative evaluation apparatus in FIG. 11.

FIG. 12B is a diagram showing an example of a screen of a notifying portion for issuing an instruction for acquiring an image in the quantitative evaluation apparatus in FIG. 11.

FIG. 12C is a diagram showing an example of a screen of the notifying portion for prompting the user to adjust the focus position in the quantitative evaluation apparatus in FIG. 11.

FIG. 12D is a diagram showing an example of a screen of the notifying portion for displaying an evaluation result of the distribution state of cells in the quantitative evaluation apparatus in FIG. 11.

FIG. 13 is a flowchart for explaining steps for evaluating the distribution state of cells by means of the quantitative evaluation apparatus in FIG. 11.

### DESCRIPTION OF EMBODIMENTS

### First Embodiment

A quantitative evaluation apparatus according to a first embodiment of the present invention will be described below with reference to the drawings.

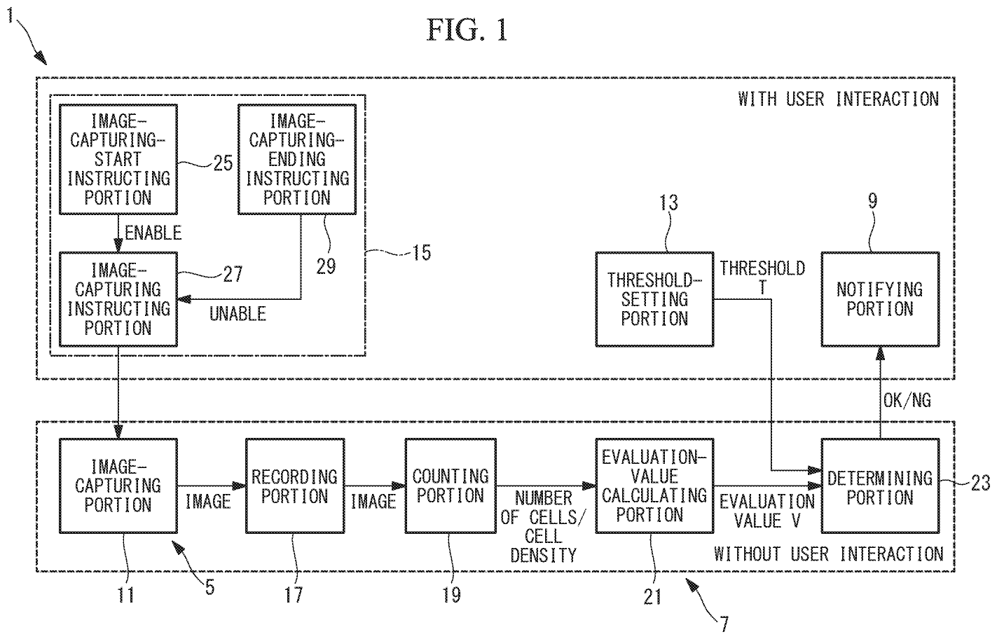

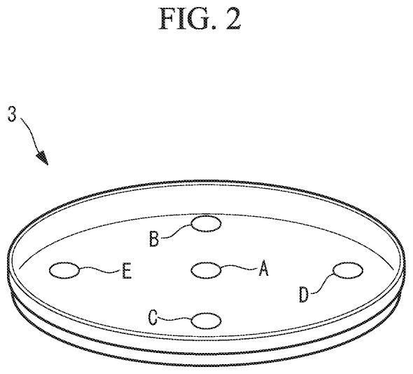

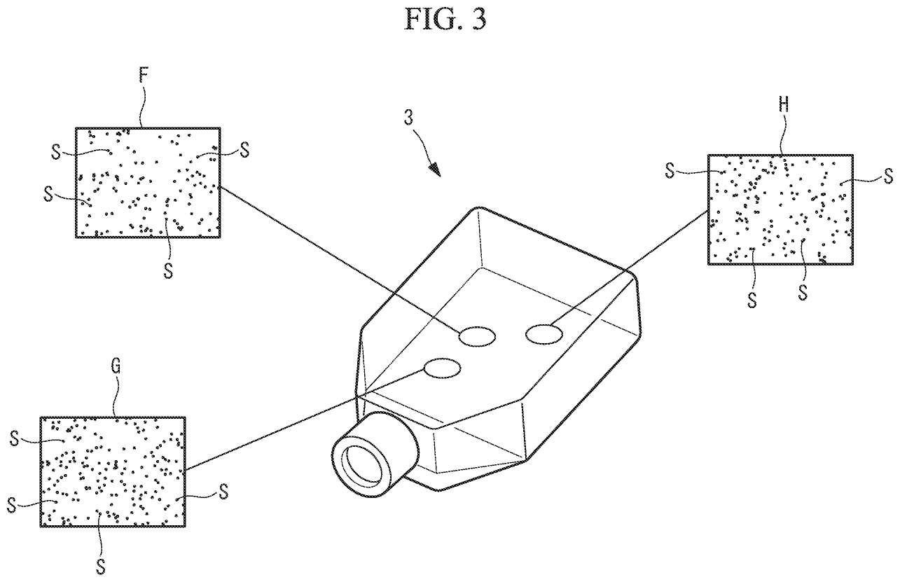

As shown in FIG. 1, a quantitative evaluation apparatus 1 according to this embodiment includes: a microscope 5 that acquires an image of the interior of a culturing vessel 3 (for example, see FIGS. 2 and 3) for culturing cells S; a PC (Personal Computer) 7 that controls the microscope 5 and processes the image acquired by the microscope 5; and a notifying portion (display) 9 such as a monitor on which the image processed by the PC 7, information, and the like is displayed.

For example, a petri dish made of an optically transparent material, as shown in FIG. 2, or a flask made of an optically transparent material, as shown in FIG. 3, is employed as the culturing vessel 3. In FIGS. 2 and 3, reference signs A, B, C, D, E, F, G, and H indicate image-capturing regions (observation positions) in the culturing vessel 3. In this embodiment, the petri dish shown in FIG. 2 will be described as an example of the culturing vessel 3. A cell suspension (not shown) is seeded in this culturing vessel 3.

The microscope 5 includes, for example: a stage (not shown) on which the culturing vessel 3 is placed; and an image-capturing portion 11 such as a camera that captures an image of the interior of the culturing vessel 3. The focus position of the image-capturing portion 11 can be adjusted by a user (operator).

As shown in FIG. 1, the PC 7 includes: a threshold-setting portion 13 with which the user sets a threshold; an image-capturing controller 15 that controls image capturing by the microscope 5; a recording portion 17 that records the image acquired by the image-capturing portion 11 of the microscope 5; a counting portion (counter) 19 that counts the numbers of cells S (seeded cells) or the densities of cells S; an evaluation-value calculating portion (evaluation-value calculator) 21 that calculates an evaluation value related to the variation (unevenness) in the distribution of the cells S in the culturing vessel 3; and a determining portion 23 that makes a determination on the evaluation value calculated by the evaluation-value calculating portion 21. The notifying portion 9, the threshold-setting portion 13, and the image-capturing controller 15 are operated by the user (components with user interaction), and the image-capturing portion 11, the recording portion 17, the counting portion 19, the evaluation-value calculating portion 21, and the determining portion 23 are not operated by the user (components without user interaction).

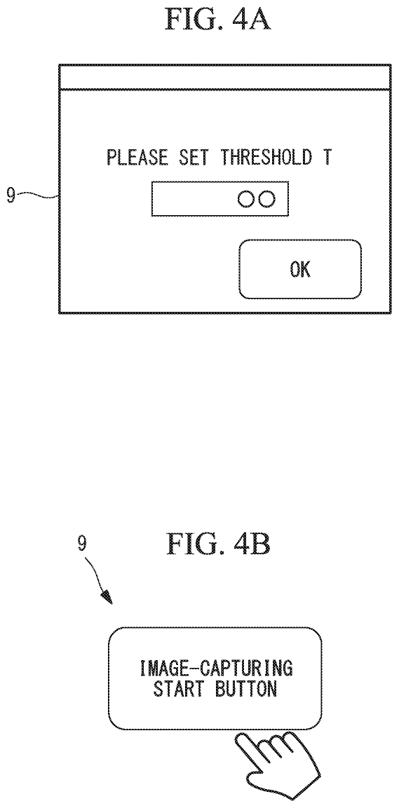

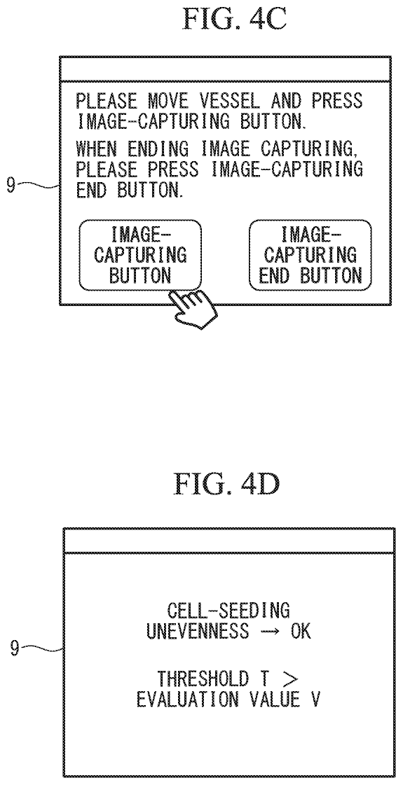

As shown in FIG. 4A, the threshold-setting portion 13 allows the user to set, on the screen of the notifying portion 9, a prescribed threshold T that indicates an acceptable degree of variation in the distribution of the cells S. As the threshold T, it is preferable to set, for example, a range for the number of cells S or the densities of cells S in the case in which the distribution of the cells S in the culturing vessel 3 is nearly uniform.

The image-capturing controller 15 includes: an image-capturing-start instructing portion 25 that sets the image capturing to a multiple-image-capturing mode in which a plurality of images are captured; an image-capturing instructing portion 27 that transmits an image-capturing instruction to the image-capturing portion 11 of the microscope 5 while the multiple-image-capturing mode is set; and an image-capturing-end instructing portion 29 that ends the multiple-image-capturing mode.

As shown in FIG. 4B, the image-capturing-start instructing portion 25 sets the image capturing to the multiple-image-capturing mode when the user presses an “image-capturing start button” displayed on the screen of the notifying portion 9.

As shown in FIG. 4C, the image-capturing instructing portion 27 causes the image-capturing portion 11 to be operated and to capture an image when the user presses an “image-capturing button” displayed on the screen of the notifying portion 9 while the multiple-image-capturing mode is set.

As shown in FIG. 4C, the image-capturing-end instructing portion 29 ends the multiple-image-capturing mode when the user presses an “image-capturing end button” displayed on the screen of the notifying portion 9 while the image capturing is set to the multiple-image-capturing mode.

The counting portion 19 reads out an image recorded in the recording portion 17 and counts the number of cells S or the density of cells S contained in the read-out image.

The evaluation-value calculating portion 21 calculates an evaluation value V on the basis of, for example, ratios of the numbers of cells S or ratios of the densities of cells S counted in images of different image-capturing regions in the culturing vessel 3. By doing so, an approximate distribution state of the cells S in the culturing vessel 3 is ascertained. With an increase in the number of image-capturing regions in the culturing vessel 3, a plurality of the evaluation values V are calculated, and thus, the distribution state of the cells S in the culturing vessel 3 is ascertained in greater detail.

The determining portion 23 determines whether or not the evaluation values V calculated by the evaluation-value calculating portion 21 are in the range of the prescribed threshold T set by the threshold-setting portion 13. In the case in which the evaluation values V for all of the image-capturing regions in the culturing vessel 3 are in the range of the prescribed threshold T, the variation of the distribution of the cells S in the culturing vessel 3 is considered to be in an acceptable range. In this case, the determining portion 23 causes the notifying portion 9 to display, as the determination result, for example, “cell-seeding unevenness→OK” on the screen of the notifying portion 9, as shown in FIG. 4D. On the other hand, in the case in which the evaluation value V of any one of the image-capturing regions in the culturing vessel 3 is outside the range of the prescribed threshold T, the variation of the distribution of the cells S in the culturing vessel 3 is considered to be outside the acceptable range. In this case, the determining portion 23 causes the notifying portion 9 to display, as the determination result, for example, “cell-seeding unevenness→unacceptable” on the screen of the notifying portion 9.

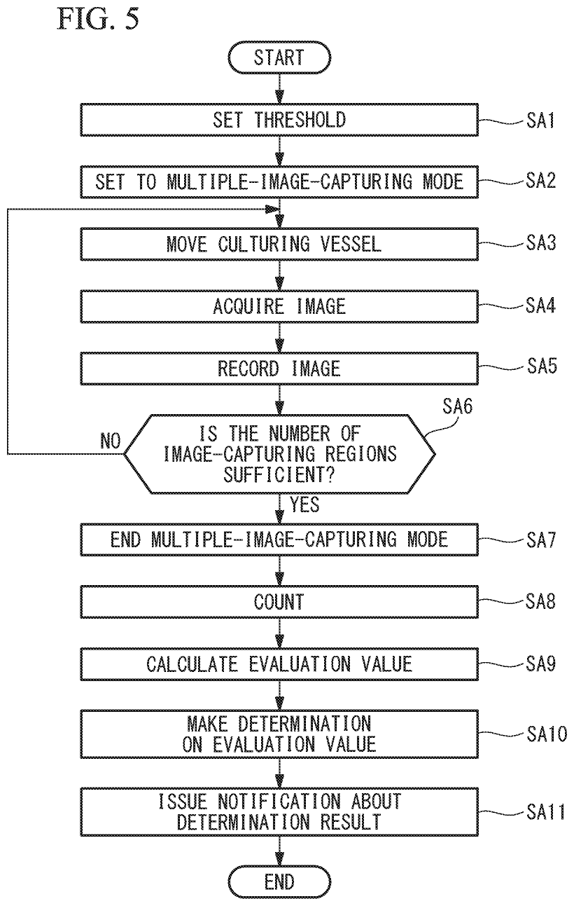

The operation of the quantitative evaluation apparatus 1, thus configured, will be described with reference to the flowchart in FIG. 5.

In order to evaluate the distribution state of the cells S in the culturing vessel 3 by means of the quantitative evaluation apparatus 1 according to this embodiment, the user places the culturing vessel 3 seeded with the cells S on the stage of the microscope 5, and, as shown in FIG. 4A, sets the threshold T by means of the threshold-setting portion 13 by inputting the prescribed threshold T on the screen of the notifying portion 9 (step SA1).

Next, as shown in FIG. 4B, the user presses the “image-capturing start button” on the screen of the notifying portion 9, thus setting the image capturing to the multiple-image-capturing mode by means of the image-capturing-start instructing portion 25 (step SA2). Next, the user moves the culturing vessel 3 on the stage of the microscope 5 so that, for example, the image-capturing region A in the culturing vessel 3 is contained in the field of view of the image-capturing portion 11 (step SA3).

Once the image-capturing region A of the culturing vessel 3 is contained in the field of view of the image-capturing portion 11, as shown in FIG. 4C, the user presses the “image-capturing button” on the screen of the notifying portion 9, thus acquiring an image of the image-capturing region A by causing the image-capturing portion 11 of the microscope 5 to be operated by means of the image-capturing instructing portion 27 (step SA4). The image acquired by the image-capturing portion 11 is transmitted to the recording portion 17 and is recorded therein (step SA5).

The user determines whether or not the number of image-capturing regions in the culturing vessel 3 is sufficient (step SA6), and repeats, also for the other image-capturing regions B, C, D, and E, the procedures in steps SA3, SA4, and SA5 for each of the image-capturing regions B, C, D, and E in the same manner as done in the image-capturing region A. Thus, images are individually acquired for the respective image-capturing regions B, C, D, and E, and recorded in the recording portion 17.

Once images of all of the image-capturing regions A, B, C, D, and E in the culturing vessel 3 are acquired, in other words, once the user determines that the number of image-capturing regions is sufficient (“YES” in step SA6), the user presses the “image-capturing end button” on the screen of the notifying portion 9 shown in FIG. 4C, thus ending the multiple-image-capturing mode by means of the image-capturing-end instructing portion 29 (step SA7).

Once the multiple-image-capturing mode is ended, the counting portion 19 reads out the images captured in the image-capturing regions A, B, C, D, and E, which are recorded in the recording portion 17, and counts the numbers of cells S or the densities of cells S in the respective images (step SA8).

Next, on the basis of the numbers of cells S or the densities of cells S counted by the counting portion 19 in each of the images, as the evaluation values V, the evaluation-value calculating portion 21 individually calculates, for example, with reference to the number of cells S or the density of cells S in the image-capturing region A, ratios of the numbers of cells S or ratios of the densities of cells S in the other image-capturing regions B, C, D, and E with respect to the number of cells S or the density of cells S in the image-capturing region A (step SA9).

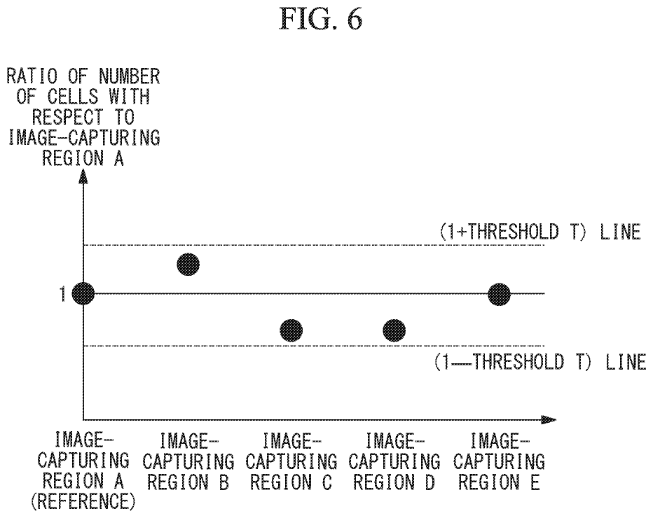

Next, by assuming that the evaluation value V of the image-capturing region A is 1, for example, as shown in FIG. 6, the determining portion 23 individually determines whether or not the evaluation values V of the other image-capturing regions B, C, D, and E with respect to the image-capturing region A are in the range of 1±threshold T (step SA10). FIG. 6 shows an example in which the ratios of the numbers of cells in the individual image-capturing regions are assumed to be the evaluation values V.

In the case in which the determining portion 23 determines that the evaluation values V of all of the image-capturing regions B, C, D, and E, as well as the evaluation value V of the image-capturing region A, are in the range of 1±threshold T, “cell-seeding unevenness→OK” is displayed on the screen of the notifying portion 9, as shown in FIG. 4D, and thus, the user is notified that the variation in the distribution of the cells S in the culturing vessel 3 is in the acceptable range.

On the other hand, in the case in which the determining portion 23 determines that even just one of the evaluation values V of the image-capturing regions A, B, C, D, and E is not in the range of 1±threshold T, “cell-seeding unevenness→unacceptable” is displayed on the screen of the notifying portion 9, and thus, the user is notified that the variation in the distribution of the cells S in the culturing vessel 3 is outside the acceptable range (step SA11).

As has been described above, with the quantitative evaluation apparatus 1 according to this embodiment, the approximate distribution state of the cells S in the culturing vessel 3 is ascertained by means of the evaluation values V calculated by the evaluation-value calculating portion 21. Then, as a result of the determining portion 23 comparing the evaluation values V with the threshold T indicating the acceptable degree of variation in the distribution of the cells S, it is possible to quantitatively determine whether or not the distribution of the cells S is nearly uniform. By doing so, it is possible to quantitatively evaluate whether or not the distribution of the cells S is uniform, and to notify the user about the evaluation result, without depending on the intuition of the user, and also without causing variations among individual users.

### Second Embodiment

Next, a quantitative evaluation apparatus according to a second embodiment of the present invention will be described below with reference to the drawings.

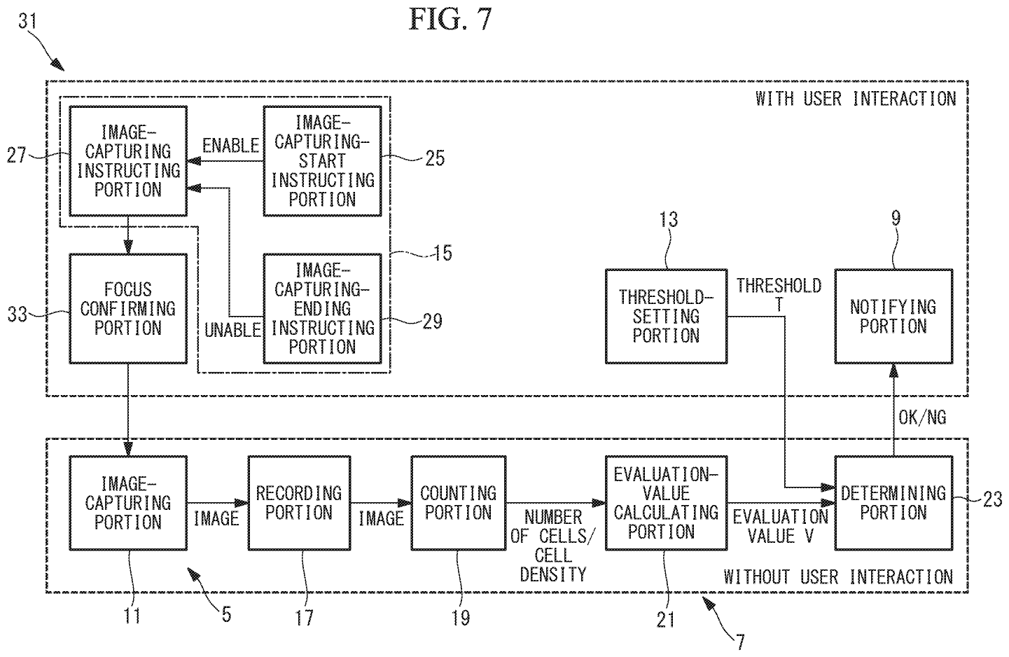

As shown in FIG. 7, a quantitative evaluation apparatus 31 according to this embodiment differs from the first embodiment in that a focus confirming portion (focus-adjustment confirming portion) 33 is included.

In describing this embodiment, portions having the same configurations as those in the quantitative evaluation apparatus 1 according to the first embodiment, described above, will be assigned the same reference signs, and descriptions thereof will be omitted.

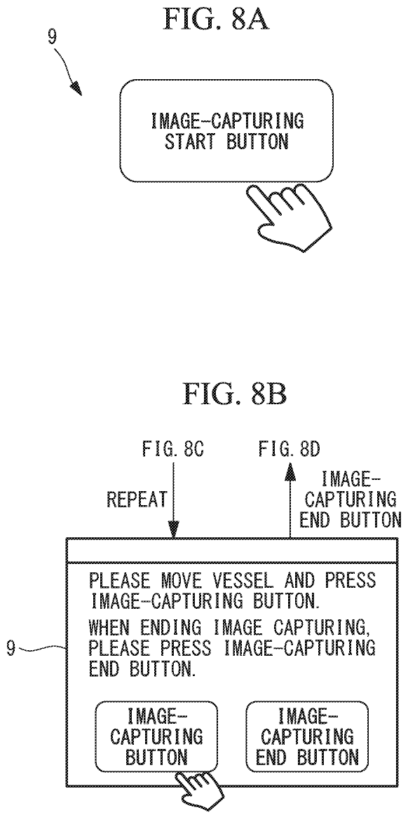

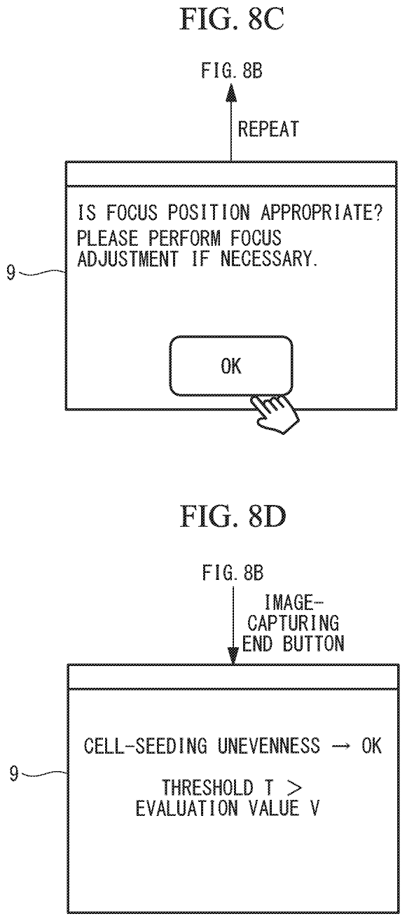

As shown in FIG. 8B, when the user presses the “image-capturing button” on the screen of the notifying portion 9 while the multiple-image-capturing mode is set, the focus confirming portion 33 prompts, on the screen of the notifying portion 9, the user to confirm whether or not to adjust the focus position of the image-capturing portion 11 of the microscope 5, as shown in FIG. 8C. The user can adjust the focus position of the microscope 5 before pressing the “OK” button on the screen of the notifying portion 9.

When the user presses the “OK” button on the screen of the notifying portion 9, the focus confirming portion 33 considers the focus position to be appropriate, and the image-capturing portion 11 is operated in accordance with the instruction from the image-capturing instructing portion 27 to perform image capturing. The focus confirming portion 33 confirms whether or not to adjust the focus position in each of the image-capturing regions.

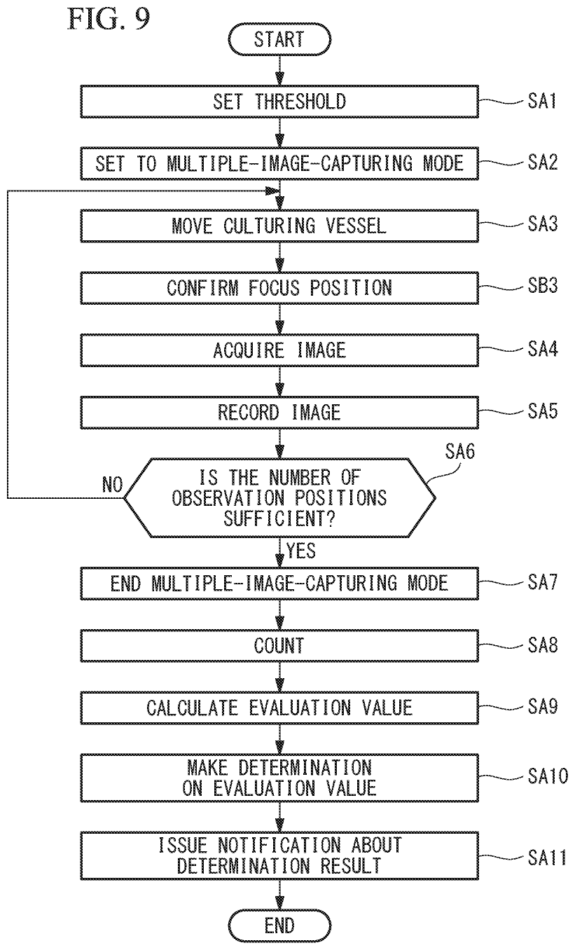

The operation of the quantitative evaluation apparatus 31, thus configured, will be described with reference to the flowchart in FIG. 9

In order to evaluate the distribution state of the cells S in the culturing vessel 3 by means of the quantitative evaluation apparatus 31 according to this embodiment, after setting the threshold T (step SA1), as shown in FIG. 8A, the user presses the “image-capturing start button” on the screen of the notifying portion 9, thus setting the image capturing to the multiple-image-capturing mode by means of the image-capturing-start instructing portion 25 (step SA2).

Then, after moving the culturing vessel 3 so that, for example, the image-capturing region A of the culturing vessel 3 is captured in the field of view of the image-capturing portion 11 (step SA3), as shown in FIG. 8B, when the user presses the “image-capturing button” on the screen of the notifying portion 9, the focus confirming portion 33 is operated, and, as shown in FIG. 8C, the user is prompted, on the screen of the notifying portion 9, to confirm whether or not to adjust the focus position of the image-capturing portion 11 in the image-capturing region A (step SB3).

Before pressing the “OK” button on the screen of the notifying portion 9 shown in FIG. 8C, the user adjusts the focus position of the image-capturing portion 11 in the image-capturing region A. Then, once the focus position is determined to be appropriate, as shown in FIG. 8C, the user presses the “OK” button on the screen of the notifying portion 9. By doing so, the image-capturing portion 11 is operated in accordance with the instruction from the image-capturing instructing portion 27 to acquire an image of the image-capturing region A (step SA4), and the acquired image is recorded in the recording portion 17 (step SA5).

The user determines whether or not the number of image capturing regions in the culturing vessel 3 is sufficient (step SA6). Then, the procedures in steps SA3, SB3, SA4, and SA5 are repeated also in the other image-capturing regions B, C, D, and E in the same manner as done in the image-capturing region A, and thus, images thereof are acquired by confirming and adjusting the focus positions in each of the image-capturing regions B, C, D, and E, and the acquired images are recorded in the recording portion 17. Regarding steps SA7 to SA11, descriptions thereof will be omitted because the procedures are the same as those in the first embodiment.

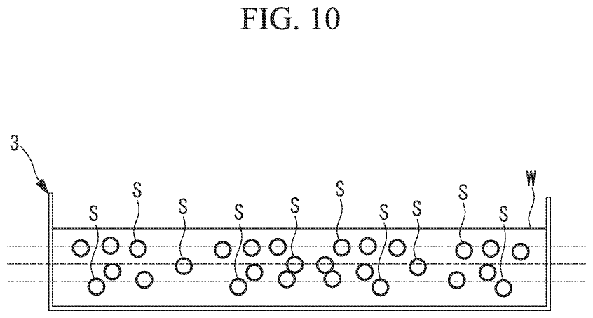

As has been described above, with the quantitative evaluation apparatus 31 according to this embodiment, for example, as shown in FIG. 10, in the case in which appropriate focus positions differ in each of the image-capturing regions A, B, C, D, and E because the cells S are suspended immediately after the cells S are seeded in the culturing vessel 3, it is possible to acquire images of the respective image-capturing regions A, B, C, D, and E at appropriate focus positions by adjusting the focus positions in each of the image-capturing regions A, B, C, D, and E. By doing so, the precisions of the evaluation values V calculated by the evaluation-value calculating portion 21 are enhanced, and thus, it is possible to quantitatively evaluate whether or not the distribution of the cells S is uniform in a more precise manner and to notify the user about the evaluation result. In FIG. 10, reference sign W indicates a culturing solution.

### Third Embodiment

Next, a quantitative evaluation apparatus according to a third embodiment of the present invention will be described below with reference to the drawings.

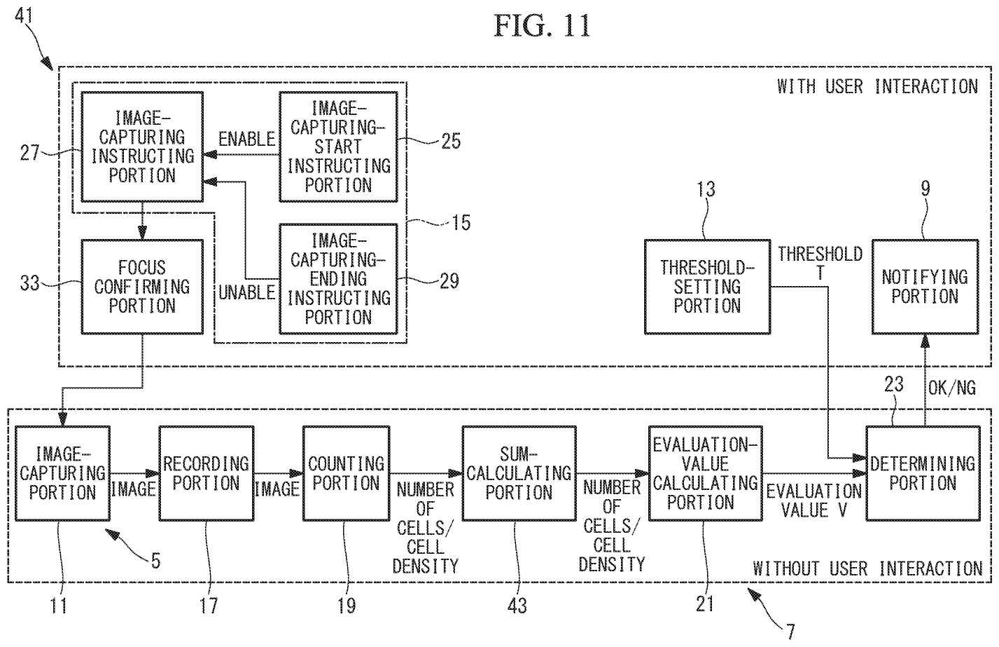

As shown in FIG. 11, a quantitative evaluation apparatus 41 according to this embodiment differs from the quantitative evaluation apparatus 31 according to the second embodiment in that a sum-calculating portion 43 is included.

In describing this embodiment, portions having the same configurations as the quantitative evaluation apparatuses 1 and 31 according to the first and second embodiments, described above, will be assigned the same reference signs, and descriptions thereof will be omitted.

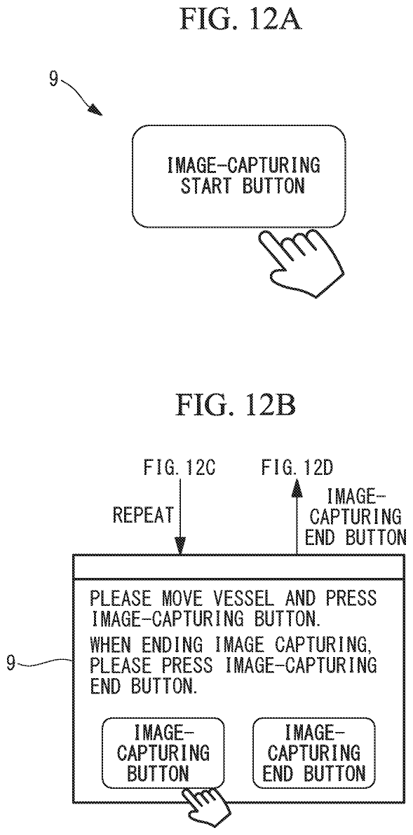

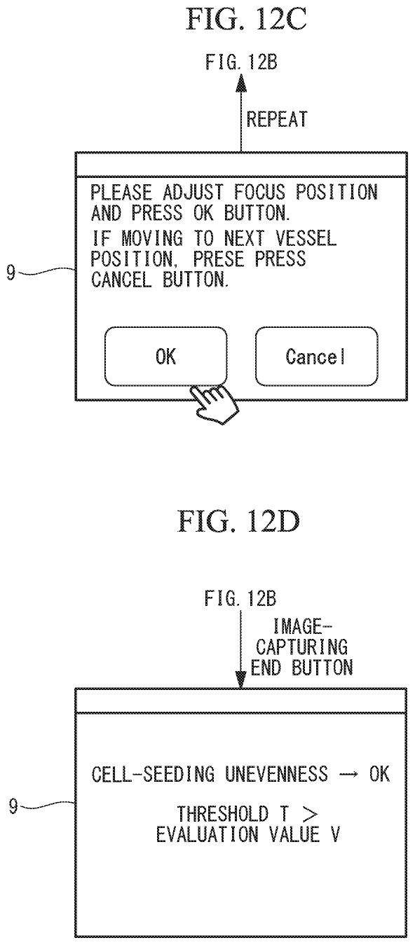

In this embodiment, as shown in FIG. 12B, when the user presses the “image-capturing button” on the screen of the notifying portion 9, as shown in FIG. 12C, the focus confirming portion 33 prompts the user, on the screen of the notifying portion 9, to adjust the focus positions in the same image-capturing regions in the culturing vessel 3. By doing so, for example, in the case in which a plurality of cells S are present at different depth positions in the same image-capturing regions, the user changes the focus positions in accordance with the individual cells S that are present at different depth positions.

As shown in FIG. 12C, when the user presses the “OK” button on the screen of the notifying portion 9, the focus confirming portion 33 considers that the focus positions have been adjusted, and the image-capturing portion 11 is operated in accordance with the instruction from the image-capturing instructing portion 27 to perform image capturing. On the other hand, when the user presses the “Cancel” button on the screen of the notifying portion 9 shown in FIG. 12C, the display returns to the screen displaying the “image-capturing button” and the “image-capturing end button” shown in FIG. 12B.

The sum-calculating portion 43 calculates the sum of the numbers of cells S or the sum of the densities of cells S counted by the counting portion 19 in the plurality of images acquired at different focus positions in the same image-capturing regions.

In this embodiment, the evaluation-value calculating portion 21 calculates the evaluation values V on the basis of the sums of the numbers of cells S or the sums of the densities of cells S calculated by the sum-calculating portion 43 for each of the image-capturing regions.

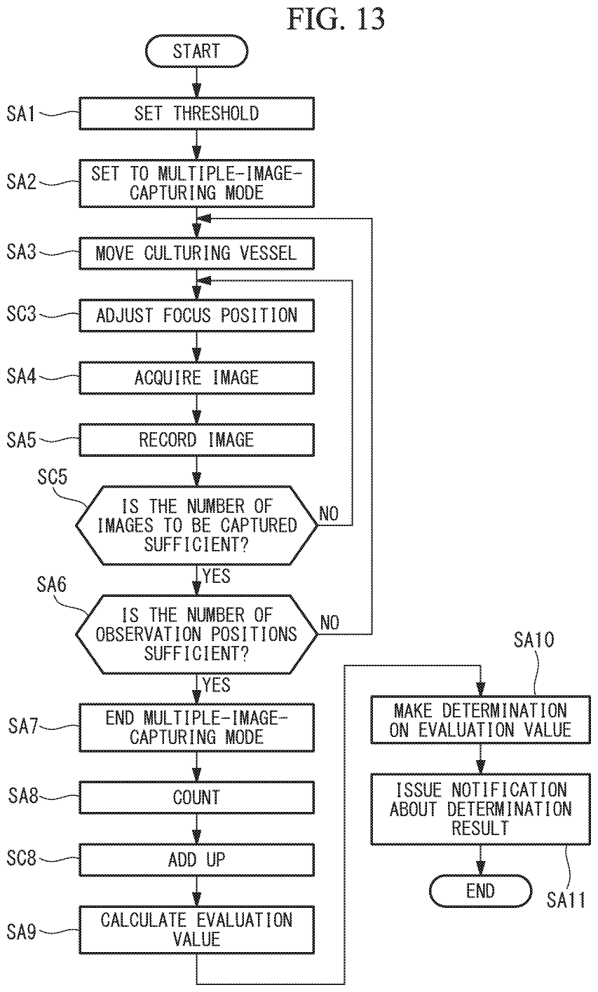

The operation of the quantitative evaluation apparatus 41, thus configured, will be described with reference to the flowchart in FIG. 13.

In order to evaluate the distribution state of the cells S in the culturing vessel 3 by means of the quantitative evaluation apparatus 41 according to this embodiment, after setting the threshold T (step SA1), as shown in FIG. 12A, the user presses the “image-capturing start button” on the screen of the notifying portion 9, thus setting the image capturing to the multiple-image-capturing mode by means of the image-capturing-start instructing portion 25 (step SA2).

Then, after moving the culturing vessel 3 so that, for example, the image-capturing region A of the culturing vessel 3 is contained in the field of view of the image-capturing portion 11 (step SA3), as shown in FIG. 12B, when the user presses the “image-capturing button” on the screen of the notifying portion 9, the focus confirming portion 33 is operated, and, as shown in FIG. 12C, the user is prompted, on the screen of the notifying portion 9, to adjust the focus position in the image-capturing region A (step SC3).

When the user adjusts the focus position in accordance with a cell S at one of the depth positions in the image-capturing region A and presses, as shown in FIG. 12C, the “OK” button on the screen of the notifying portion 9, the image-capturing portion 11 is operated in accordance with the instruction from the image-capturing instructing portion 27 to capture an image at the changed focus position in the image-capturing region A (step SA4), and the acquired image is recorded in the recording portion 17 (step SA5).

The user determines whether or not the number of images captured in the image-capturing region A of the culturing vessel 3 is sufficient (step SC5), and, in the case in which the number of images captured in the image-capturing region A is determined to be insufficient, the procedure returns to step SC3. Then, steps SC3, SA4, and SA5 are repeated in the image-capturing region A, the focus position is adjusted in accordance with another cell S at a different depth position, and an image is acquired.

These procedures are repeated in the image-capturing region A, images of the plurality of cells S present at different depths are acquired without missing any of the cells S, and the acquired images are recorded. Then, in the case in which the user determines that the number of images captured in the image-capturing region A is sufficient, the user presses the “Cancel” button on the screen of the notifying portion 9 shown in FIG. 12C, thus returning to the screen displaying the “image-capturing button” and the “image-capturing end button” shown in FIG. 12B.

Next, the user determines whether or not the number of image-capturing regions in the culturing vessel 3 is sufficient (step SA6). Then, the procedures in steps SA3, SC3, SA4, and SA5 and SC5 are repeated also in the other image-capturing regions B, C, D, and E in the same manner as done in the image-capturing region A, and thus, while changing the focus positions to multiple locations in each of the image-capturing regions B, C, D, and E, images thereof at the plurality of focus positions are acquired by confirming and adjusting the focus positions, and the acquired images are recorded in the recording portion 17.

Once images of all of the image-capturing regions A, B, C, D, and E in the culturing vessel 3 are acquired, the user presses the “image-capturing end button” on the screen of the notifying portion 9 shown in FIG. 12B, thus ending the multiple-image-capturing mode by means of the image-capturing-end instructing portion 29 (step SA7). Once the multiple-image-capturing mode is ended, the counting portion 19 reads out the images captured in the image-capturing regions A, B, C, D, and E, which are recorded in the recording portion 17, and the numbers of cells S or the densities of cells S in the respective images are counted (step SA8).

Next, the sum-calculating portion 43 adds the numbers of cells S or the densities of cells S individually counted in the plurality of images captured at different focus positions in each of the image-capturing regions A, B, C, D, and E, thus calculating the sums for each of the respective image-capturing regions A, B, C, D, and E (step SC8).

Next, the evaluation-value calculating portion 21 calculates the evaluation values V on the basis of the sums of the numbers of cells S or the sums of the densities of cells S of the respective image-capturing regions A, B, C, D, and E calculated by the sum-calculating portion 43 (step SA9). Regarding steps SA10 and SA11, descriptions thereof will be omitted because the procedures are the same as in the first embodiment.

As has been described above, with the quantitative evaluation apparatus 41 according to this embodiment, as shown in FIG. 10, in the case in which all of the cells S are not contained in images captured at one focus position in the individual image-capturing regions because the cells S are suspended immediately after the cells S are seeded in the culturing vessel 3, in accordance with the reminder for adjusting the focus positions, given by the focus confirming portion 33, the user changes the focus positions in each of the image-capturing regions and acquires images at a plurality of focus positions, and thus, it is possible to acquire images of the plurality of cells S present in the respective image-capturing regions without missing any of the cells S. Thus, by means of the evaluation-value calculating portion 21, it is possible to calculate the evaluation values V in a precise manner on the basis of the sums of the numbers of cells S or the sums of the densities of cells S in the images acquired at the plurality of focus positions in the same image-capturing regions. Therefore, it is possible to quantitatively evaluate whether or not the distribution of the cells S is uniform in a more precise manner and to notify the operator about the evaluation result.

Although this embodiment is assumed to include the sum-calculating portion 43 that calculates the sums of the numbers of the cells S or the sums of the densities of cells S counted, by means of the counting portion 19, in the images acquired at the plurality of different focus positions in the same image-capturing regions, alternatively, an average calculating portion may be included, the average calculating portion calculating the averages of the numbers of cells S or the averages of the densities of cells S, counted by means of the counting portion 19, in the images acquired at the plurality of different focus positions in the same image-capturing regions.

As above, although the embodiments of the present invention have been described in detail with reference to the drawings, specific configurations are not limited to these embodiments, and design alterations or the like that do not depart from the scope of the present invention are also encompassed. For example, without limitation to the forms in which the present invention is applied to the above-described embodiments and modifications, the present invention may be applied to embodiments in which these embodiments and modifications are combined, as appropriate, and it is not particularly limited.

For example, the evaluation-value calculating portion 21 may calculate the evaluation values V on the basis of standard deviations of the numbers of cells S or standard deviations of the densities of cells S counted in the images of different image-capturing regions.

By doing so, it is possible to ascertain the distribution state of the cells S in each of the image-capturing regions. With an increase in the number of image-capturing regions, it is possible to ascertain the distribution state of the cells S in the culturing vessel 3 in greater detail.

For example, the evaluation-value calculating portion 21 may calculate an evaluation value V on the basis of a difference between a maximum value and a minimum value of the numbers of the cells S or a difference between a maximum value and a minimum value of the densities of the cells S counted in the images of different image-capturing regions.

By doing so, it is possible to ascertain the approximate distribution state of the cells S in the culturing vessel 3 in a simple manner.

As a result, the following aspect is read from the above described embodiment of the present invention.

An aspect of the present invention is a quantitative evaluation apparatus including: a counter that counts numbers of cells or densities of cells contained in individual images of a plurality of image-capturing regions in a culturing vessel for culturing the cells; an evaluation-value calculating portion that calculates an evaluation value related to variation in distribution of the cells in the culturing vessel on the basis of the numbers of the cells or the densities of the cells counted by means of the counter in each of the images; a determining portion that determines whether or not the evaluation value calculated by the evaluation-value calculating portion is in a range of a prescribed threshold; and a display that displays determination result determined by the determining portion.

With this aspect, as a result of calculating, by means of the evaluation-value calculating portion, the evaluation value related to the variation in the distribution of the cells in the culturing vessel on the basis of the numbers of cells or the densities of cells counted by means of the counting portion in each of the images of the plurality of image-capturing regions in the culturing vessel, an approximate distribution state of the cells in the culturing vessel is ascertained.

Therefore, by setting in advance, as the threshold, the range for the number of cells or the densities of cells in the case in which the distribution of the cells in the culturing vessel is nearly uniform, it is possible to quantitatively determine, by means of the determining portion, whether or not the distribution of the cells is uniform, and to notify the operator about the determination result by means of the display portion. By doing so, it is possible to quantitatively evaluate whether or not the distribution of the cells is uniform, and to notify the operator about the evaluation result without depending on the intuition of the operator, and also without causing variations among individual operators.

In the above-described aspect, the evaluation-value calculating portion may calculate the evaluation value on the basis of a ratio of the numbers of the cells or a ratio of the densities of cells counted in the images of the different image-capturing regions.

By employing such a configuration, it is possible to ascertain the distribution state of the cells in each of the image-capturing regions. With an increase in the number of the image-capturing regions, the distribution state of the cells in the culturing vessel is ascertained in greater detail.

In the above-described aspect, the evaluation-value calculating portion may calculate the evaluation value on the basis of a standard deviation of the numbers of the cells or a standard deviation of the densities of cells counted in the images of the different image-capturing regions.

By employing such a configuration, it is possible to ascertain the distribution state of the cells in each of the image-capturing regions. With an increase in the number of the image-capturing regions, the distribution state of the cells in the culturing vessel is ascertained in greater detail.

In the above-described aspect, the evaluation-value calculating portion may calculate the evaluation value on the basis of a difference between a maximum value and a minimum value of the numbers of the cells or a difference between a maximum value and a minimum value of the densities of cells counted in the images of the different image-capturing regions.

By employing such a configuration, it is possible to ascertain, in a simple manner, an approximate distribution state of the cells in the culturing vessel.

The above-described aspect may further include: an image-capturing portion that is capable of adjusting a focus position; and a focus-adjustment confirming portion that prompts an operator to confirm whether or not to adjust the focus position of the image-capturing portion in each of the image-capturing regions.

In some cases, the cells are suspended immediately after the cells are seeded in the culturing vessel, and thus, the optimal focus position differs in each of the image-capturing regions. Therefore, by employing such a configuration, it is possible for the operator to determine whether or not to adjust the focus position of the image-capturing portion by means of the focus-adjustment confirming portion, and to acquire images acquired at the optimal focus positions of the respective image-capturing regions. By doing so, it is possible to enhance the precision of the evaluation value calculated by the evaluation-value calculating portion.

In the above-described aspect, the focus-adjustment confirming portion may prompt the operator to adjust the focus position in the same image-capturing regions, and the evaluation-value calculating portion may calculate the evaluation value on the basis of a sum of the numbers of the cells or the sum of the densities of cells or, an average of the numbers of the cells or an average of the densities of cells counted in the images acquired at the different focus positions in the same image-capturing regions.

In some cases, the cells are suspended immediately after the cells are seeded in the culturing vessel, and thus, all of the cells are not included in an image captured at one focus position in each of the image-capturing regions. Therefore, by employing such a configuration, as a result of prompting the operator to adjust the focus position by means of the focus-adjustment confirming portion, it is possible for the operator to acquire the plurality of images while changing the focus position of the image-capturing portion in the same image-capturing regions, and to acquire images of the plurality of the cells present in the respective image-capturing regions without missing any of the cells.

Also, it is possible to calculate the evaluation value by means of the evaluation-value calculating portion in a precise manner on the basis of the sum of the numbers of cells or the sum of the densities of cells or, the average of the numbers of cells or the average of the densities of cells in each of the images in the same image-capturing regions. By doing so, it is possible to quantitatively evaluate whether or not the distribution of the cells is uniform in a more precise manner, and to notify the operator about the evaluation result.

### REFERENCE SIGNS LIST

1, 31, 41 quantitative evaluation apparatus
9 notifying portion (display)
11 image-capturing portion
19 counting portion (counter)
21 evaluation-value calculating portion (evaluation-value calculator)
23 determining portion
33 focus confirming portion (focus-adjustment confirming portion)
S cell

1. A quantitative evaluation apparatus comprising:
a counter that is configured to count numbers of cells or densities of cells contained in individual images of a plurality of image-capturing regions in a culturing vessel for culturing the cells;
an evaluation-value calculator that is configured to calculate an evaluation value related to variation in distribution of the cells in the culturing vessel on the basis of the numbers of the cells or the densities of the cells counted by means of the counter in each of the images;
a determining portion that is configured to determine whether or not the evaluation value calculated by the evaluation-value calculator is in a range of a prescribed threshold; and
a display that is configured to display determination result determined by the determining portion.

2. A quantitative evaluation apparatus according to claim 1, wherein the evaluation-value calculator calculates the evaluation value on the basis of a ratio of the numbers of the cells or a ratio of the densities of the cells counted in the images of the different image-capturing regions.

3. A quantitative evaluation apparatus according to claim 1, wherein the evaluation-value calculator calculates the evaluation value on the basis of a standard deviation of the numbers of the cells or a standard deviation of the densities of the cells counted in the images of the different image-capturing regions.

4. A quantitative evaluation apparatus according to claim 1, wherein the evaluation-value calculator calculates the evaluation value on the basis of a difference between a maximum value and a minimum value of the numbers of the cells or a difference between a maximum value and a minimum value of the densities of the cells counted in the images of the different image-capturing regions.

5. A quantitative evaluation apparatus according to claim 1, further comprising:
an image-capturing portion that is configured to adjust a focus position; and
a focus-adjustment confirming portion that is configured to prompt an operator to confirm whether or not to adjust the focus position of the image-capturing portion in each of the image-capturing regions.

6. A quantitative evaluation apparatus according to claim 5, wherein the focus-adjustment confirming portion prompts the operator to adjust the focus position in the same image-capturing regions, and
the evaluation-value calculator calculates the evaluation value on the basis of a sum of the numbers of the cells or a sum of the densities of the cells or, an average of the numbers of the cells or an average of the densities of the cells counted in the images acquired at the different focus positions in the same image-capturing regions.
